(19) United States
(12) Patent Application Publication
(10) Pub. No.: US 2024/0122654 A1
(43) Pub. Date: Apr. 18, 2024

(54) DEVICE FOR USE IN COMPUTER-AIDED SURGERY

(71) Applicants: Medos International Sarl, LeLocle (CH); eCential Robotics, Gières (FR)

(72) Inventors: William Frasier, New Bedford, MA (US); Thomas Kuenzi, Magden (CH); Philippe Lindenmann, Basal (CH); Michael White, Oberdorf (CH); Marc Puls, Thörigen (CH); Timothy Minogue, Duxbury, MA (US); Montserrat Ruth Charles-Harris Ferrer, Leymen (FR); Sharmila Palani, Liestal (CH); Clement Vidale, Meylan (FR); Stephane Lavallee, Saint Martin d Uriage (FR); Paul Mesple, Grenonble (FR)

(21) Appl. No.: 18/047,553

(22) Filed: Oct. 18, 2022

(51) Int. Cl. A61B 34/20

(52) U.S. Cl. CPC A61B 34/20; A61B 2034/2055; A61B 2034/207

(57) ABSTRACT

A device for computer aided surgery, including: a body; an optical element coupled to the body; a fiducial coupled to the body; and a calibration element. The calibration element be a pointer tip or a divot, either individually or in combination.

### TECHNICAL FIELD

Various exemplary embodiments disclosed herein relate generally to devices for use in computer-aided surgery (CAS).

### BACKGROUND

The processes of registration and calibration in computer-aided surgery typically use a number of separate, discrete components.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a device for computer aided surgery, comprising: a body; an optical element coupled to the body; a fiducial coupled to the body; and a calibration element.

Various embodiments are described, wherein the device comprises at least three optical elements.

Various embodiments are described, wherein the device comprises at least three fiducials.

Various embodiments are described, wherein the calibration element is a calibration divot forming an indentation on the body configured to receive a surgical instrument.

Various embodiments are described, wherein the calibration divot further comprises a flat cylindrical positioning divot.

Various embodiments are described, wherein the calibration divot further comprises a conical positioning divot.

Various embodiments are described, wherein the calibration element is a pointer tip extending from the body.

Various embodiments are described, wherein the pointer tip is adapted to receive a first pointer extension.

Various embodiments are described, wherein the pointer tip is removable from the body.

Various embodiments are described, wherein the pointer tip is affixed to a pointer extension on the body.

Various embodiments are described, further including a second pointer extension having a different length than the first pointer extension.

Various embodiments are described, further including data representing an as-manufactured dimension of the device.

Further various embodiments relate to a device for computer aided surgery, comprising: a body; an optical element, wherein the optical element is configured to be visible to a location camera; and a fiducial, wherein the fiducial is radiopaque; and a calibration element, wherein the optical element, the fiducial, the body, and the calibration element are held in relation to each other in a fixed position.

Various embodiments are described, wherein the calibration element is a calibration divot forming an indentation on the body configured to receive a surgical instrument.

Various embodiments are described, wherein the calibration divot further comprises a flat cylindrical positioning divot.

Various embodiments are described, wherein the calibration divot further comprises a conical positioning divot.

Various embodiments are described, wherein the calibration element is a pointer tip mounted to the body.

Various embodiments are described, wherein the pointer tip is configured to receive a first pointer extension.

Various embodiments are described, wherein the pointer tip is removable from the body.

Various embodiments are described, wherein the pointer tip is affixed to a pointer extension on the body.

Various embodiments are described, further including a second pointer extension having a different length than the pointer extension.

Various embodiments are described, further including data representing an as-manufactured dimension of the device.

### BRIEF DESCRIPTION OF THE DRAWINGS

In order to better understand various exemplary embodiments, reference is made to the accompanying drawings, wherein embodiments of a device for use in computer-aided surgery are shown:

FIG. 1 is a front perspective view of a device for use in computer-aided surgery;

FIG. 2 is a rear perspective view of the device of FIG. 1;

FIG. 3 is an exploded view of the device of FIG. 1;

FIG. 4 is a top view of the device of FIG. 1;

FIG. 5 is a right side view of the device of FIG. 1;

FIG. 6 is a bottom view of the device of FIG. 1;

FIG. 7 is a front view of the device of FIG. 1;

FIG. 8 is back view of the device of FIG. 1;

FIG. 9 is a bottom perspective view of the device of FIG. 1;

FIG. 10 is a top view of another device for use in computer-aided surgery;

FIG. 11 is a right side view of the device of FIG. 10;

FIG. 12 is a back side view of the device of FIG. 10;

FIG. 13 is a top perspective view of the device of FIG. 10; and

FIG. 14 is a top perspective view of the device of FIG. 10.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Before computer-aided surgery (CAS) surgery takes place, the CAS system learns the locations and relationships of various elements like the patient (based on images of the patient which might be obtained by a fluoroscopy, x-ray, CT, MRI, etc.) and medical instruments (e.g., scalpel, saw, drill, bone screw, implant, robot, etc.). To enable the CAS to locate the patient, the patient typically has a navigation array attached somewhere on their body, often attached to a bone for stability. These navigation arrays can be monitored by a location device or system such as a spatial camera, one of which is commercially available from Northern Digital Inc. Spatial cameras typically use an internal coordinate system that is defined by the camera, not by the location of the patient (the spatial camera can be placed in various locations relative to the patient). The navigation arrays may be an array of reflective spheres that reflect light back to the spatial camera (the spatial camera or other light source might emit infrared (IR) light and then sense the IR light reflected back from the spheres using stereoscopic cameras, and thereby being able to spatially locate the spheres). Alternatively, the navigation arrays can be LEDs (or other point light sources) that emit light that will be sensed by the spatial camera (no reflection is required). Further, instead of navigation arrays and a spatial camera, the spatial system might use electromagnetic devices that emit signals that can be used to determine their spatial location by a receiver, or other known systems for navigation of devices.

Many surgeries use imaging devices (e.g., fluoroscope, x-ray, CT, MRI) that take images of the patient which can be helpful to the surgeon during surgery. Fiducials, such as radiopaque markers, can be attached to the patient before the imaging occurs. These fiducials make relatively well-defined landmarks in the image which can be used later to transform between the patient coordinate system and the camera coordinate system. The imaging devices typically have their own internal coordinate system that is defined by the imaging device itself and has no fixed relation to the coordinate system of the spatial camera (the camera can typically be placed in various locations relative to the imaging device).

Navigation arrays can also be attached to surgical instruments so that the CAS system can track the spatial location of the instrument. The spatial camera tracks the location of the navigation array, and thus the surgical instrument in the coordinate system of the camera. But it is only part of the picture for the spatial camera to know the location of the surgical instrument in the camera coordinate system. It is helpful for the CAS system to be able to know where the instrument is relative to the patient.

To accomplish this, various processes are used in setting up the CAS system before a surgery. One process is used to allow the CAS system to harmonize between the spatial camera coordinate system, the patient coordinate system, and the image device coordinate system—this process is typically called registration. In registration, the CAS system determines the relationship between the various coordinate systems. That is, if the CAS system knows the spatial relationship between navigation arrays connected to the patient (which are monitored by the spatial camera) and the fiducials connected to the patient (which show up in the images created by the imaging device), the CAS system can relate that information mathematically/spatially so that the image of the patient can be appropriately aligned with or overlaid onto the patient in 3D space.

The CAS system also needs to know the spatial relationship between the navigation array and the tip of the surgical instrument, as the tip is the part that may be altering the tissue of the patient. Another process is used to allow the CAS to obtain this relationship—this is typically called calibration. The term calibration may be used to describe the scenario in which the CAS system learns the distance or geometric relationship between the array and the tip of the tool, for example when the CAS system does not know the exact geometry of the surgical instrument. If the CAS system allows any length saw blade to be used, it can require the user to calibrate the tip of the blade. To accomplish this, the CAS system can use a “pointer” which is another surgical instrument with a pointed tip, a shaft, and a navigation array connected to the shaft such that the tip is located at a fixed location relative to the array. The CAS system is programmed to know this fixed geometric relationship and thus can use the pointer to obtain geometric points in 3D space, such as the tip of the saw blade (other points on the saw blade may be used such as divots on the saw blade that have a known relation to the tip of the saw blade) and can then deduce the relationship between the tip of the saw blade and the navigation arrays.

In some scenarios, the CAS system might require that only a certain length saw blade be used (and knows the length and geometry of that expected saw blade sometimes referred to as pre-calibrated instruments). In such as case, the CAS system may perform what is typically called a calibration verification. In a calibration verification, the user touches the tip of the pointer to the tip of the saw blade (or to a divot as noted above) and the CAS system determines if the tip of the pointer is where it expects the tip of the saw blade (or divot) to be spatially located.

These processes harmonize the spatial relationships between the various elements of the CAS system. In this manner, the CAS system can know where the tip of the saw blade is relative to the patient, not just relative to the camera system, and images can be correlated to the actual position of the patient providing surgeons with information not available to the eye, such as the locations of bone or even nerves whose view is obstructed by the patient's skin. Conventional systems often used separate devices for the navigation arrays, the fiducials, and the pointers. Such systems created additional inaccuracies and complication in the surgical procedure, e.g., grabbing for multiple elements during various stages of the surgical flow.

An exemplary embodiment of a device for use in computer-aided surgery will be described that combines some or all of reflective spheres, radiopaque fiducials, a pointer, and a divot into one apparatus. This reduces the number of devices needed for registration and/or calibration and therefore may streamline these processes and may simplify the surgical workflow. The pointer and the divots may be called calibration elements, either individually or in combination.

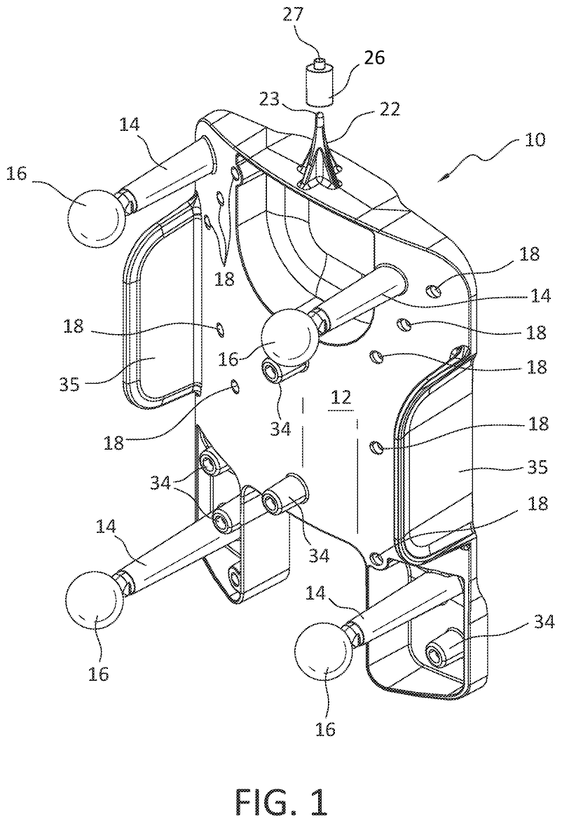

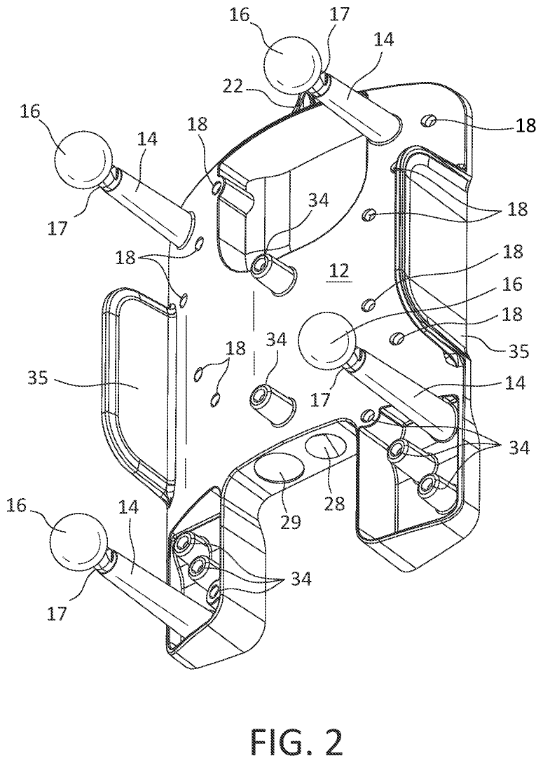

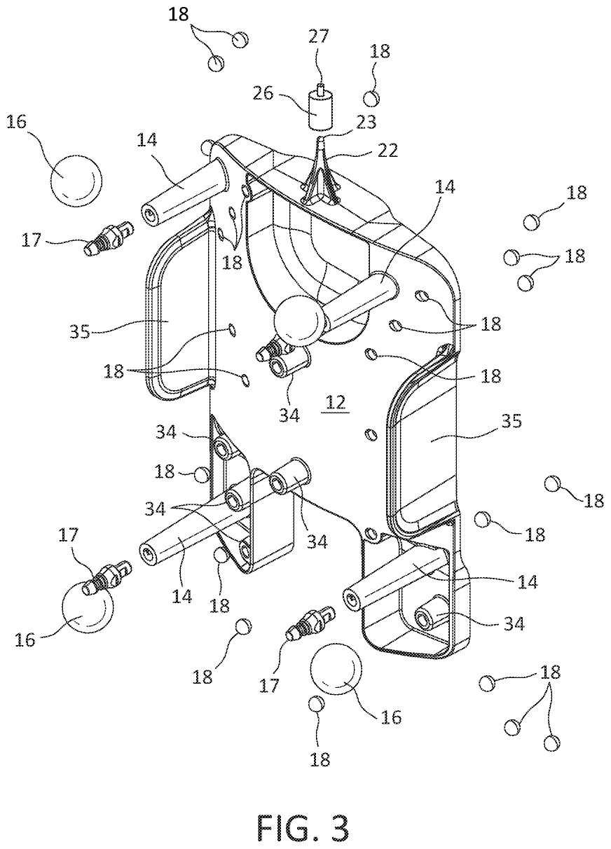

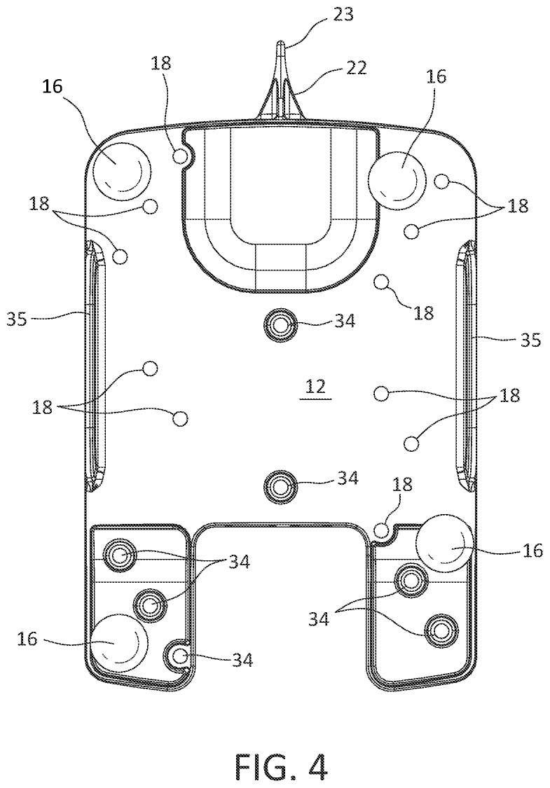

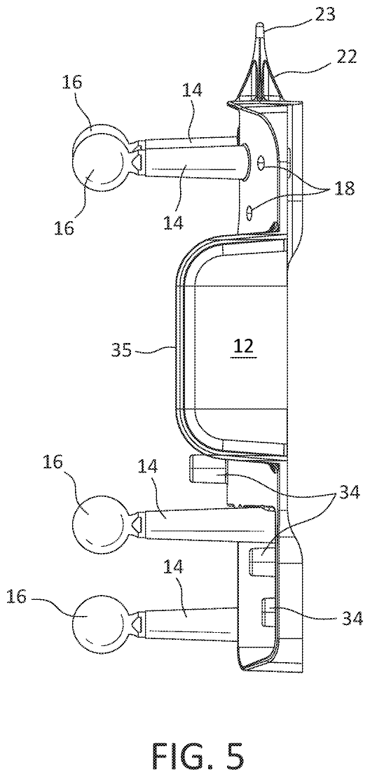

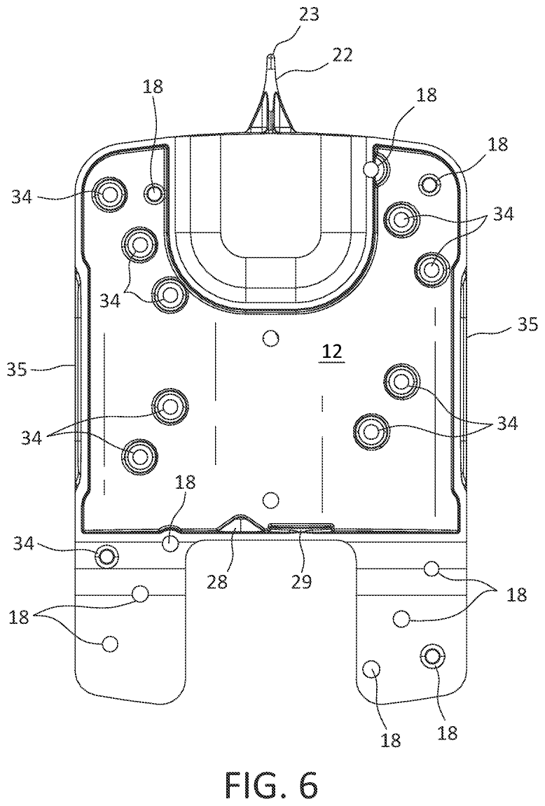

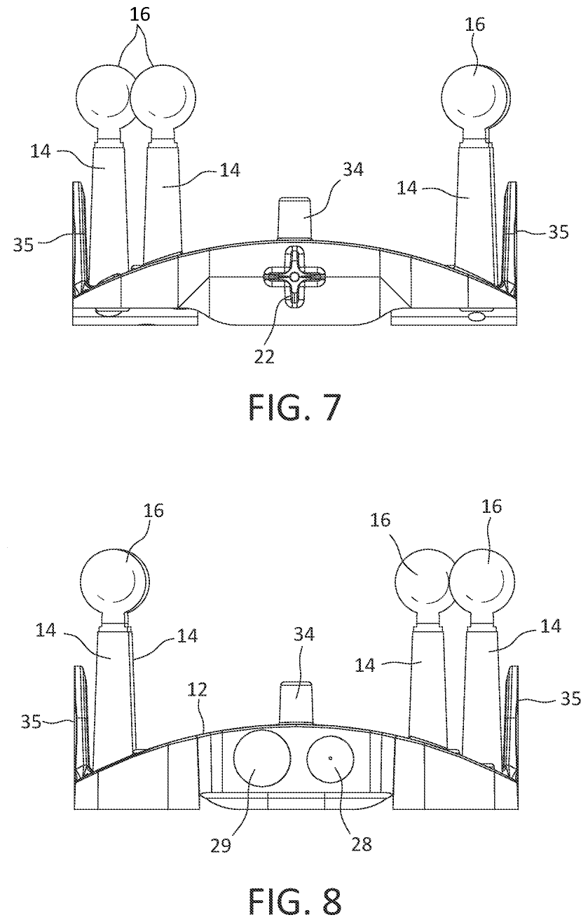

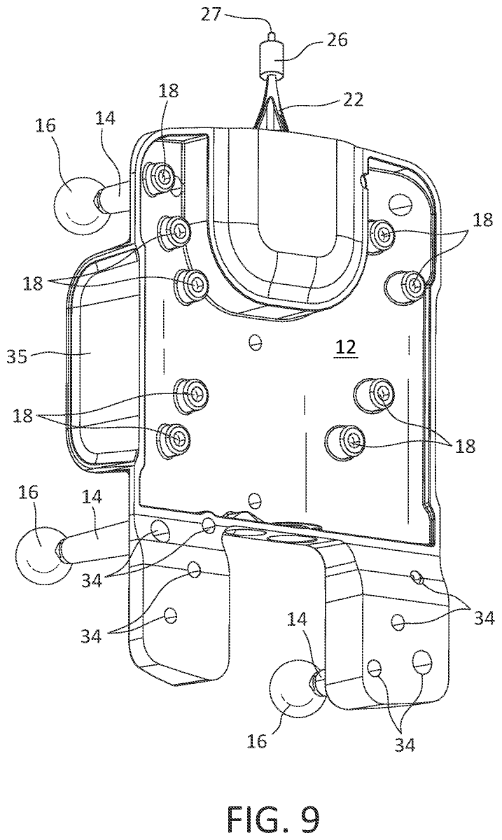

FIGS. 1 through 9 illustrate an exemplary embodiment of a device for use in computer-aided surgery.

FIGS. 1 through 9 illustrate a device 10 including a body 12. The body 12 has four posts 14, with each respective post supporting a reflective sphere 16. The reflective spheres 16 are optically visible to the camera. The reflective spheres thus provide an optical element.

Although the posts 14 are shown as elongated conical cylinders, they may be of any shape, and have different lengths from each other. The posts 14 may simply be mounting locations on the body 12.

The posts 14 are injection molded with the body 12. Although illustrated as being injection molded to be integral with the body 12, the posts 14 may be separate components mounted by any method including being attached by snap fit, threaded fit, a separate fastener, or a collet feature. Although the posts 14 are shown as elongated conical cylinders, they may be of any shape. Also, different posts 14 may be of different lengths from each other or the same length. In this embodiment the posts 14 are of the same length so the reflective spheres 16 are in a horizontal plane. The reflective spheres 16 form an optically visible array. The reflective spheres 16 may be attached to the posts 14 by snap-on features 17, but other attachment mechanisms and methods may also be used. The reflective spheres 16 provide an optical element. While reflective spheres 16 are used as an example of an optical element, the optical element may take other shapes as well. The reflective spheres 16 are an example of a passive optical element. In other embodiments, the optical elements may instead be active optical elements that may include precise light sources, for example light emitting diodes (LEDs), that emit light that is captured by the camera and then used to determine the location of the optical elements. With knowledge of the spatial location of the optical elements, the CAS system can then determine, using a priori knowledge of the spatial relationship of the various features of device 10 to each other, the spatial location of those features, such as fiducials 18 which will be described in more detail below.

The posts 14 each support a respective optically reflective sphere 16. The reflective spheres 16 form an optically visible array. These are illustrated as being connected to the end of a post 14 with a snap-on feature. The purpose of the respective spheres 16 is to be visible to a location device such as a spatial camera. The fiducials 18 provide marks that show up on images taken by an imaging device (such as fluoroscope or X-ray). The marks can be used to determine location information relative to the patient. Also, the device may include a divot that may be used to calibrate an instrument as described above. The device 10 holds reflective spheres 16, fiducials 18, pointer extension post 22 with pointer tip 23, and divots 28, 29 in a fixed spatial relationship. The CAS system will be programmed to know this fixed geometric relationship, and thus can use that information beneficially during registration and/or calibration.

The body 12 is shown as having four reflective spheres (or optical elements). It is noted however that more than four spheres can be used and that three spheres are typically sufficient. The reflective spheres 16 may be optical elements that have other shapes, for example having a cubic, elongated tip, or paddle shape. The reflectivity of the optical elements can be partially reflective or fully reflective. The reflectivity may be achieved during manufacturing using a paint, coating, impregnation, or other technique. Also, the optical elements may be molded as an integral part of the posts 14.

The fiducials 18 are typically molded into the body 12 but may also be attached to the body 12 using a snap fit or fasteners. The fiducials 18 are radiopaque and are visible during, for example, a C-arm X-ray (CBCT) scan of the patient while the device 10 is placed on or attached to the patient. The fiducials 18 may be separate components mounted by any method including being attached by snap fit, threaded fit, a separate fastener, a collet feature, or overmolded. By radiopaque it is intended that the fiducials 18 are detectable from surrounding material by X-ray (for other imaging devices, the fiducials may be any other material that would show clearly in the images taken). Accordingly, the fiducials 18 may be partially transmissive and partially translucent to X-rays, so that the location of the fiducials 18 can be obtained by processing the X-ray images after an X-ray scan of the patient is taken with the device 10 placed on or attached to the patient.

Because the CAS system knows the spatial relationship of reflective spheres 16, fiducials 18, pointer tip 23, and divots 28, 29, the CAS system can then calculate the location and orientation of the fiducials 18, pointer tip 23, and divots 28, 29. The fixed relationship between reflective spheres 16 and fiducials 18 can be used in the registration process described above.

Device 10 also includes a pointer extension post 22. The pointer extension post 22 may be integrally formed with body 12 or may be attachable to body 12. The CAS system needs to know the spatial relationship of the features of the device relative to the tip 27. As such, the CAS system must know whether a pointer extension has been attached to the device 10. This knowledge can be achieved via a configuration GUI screen or may be achieved by a machine vision system that evaluates an image of the device 10 and determines from image analysis whether a pointer extension is attached. Alternatively, the device 10 can include electronics such as RFID sensors, proximity sensors to determine if and possibly which length pointer extension has been attached. The device 10 can include wireless communications to communicate this information to the CAS system.

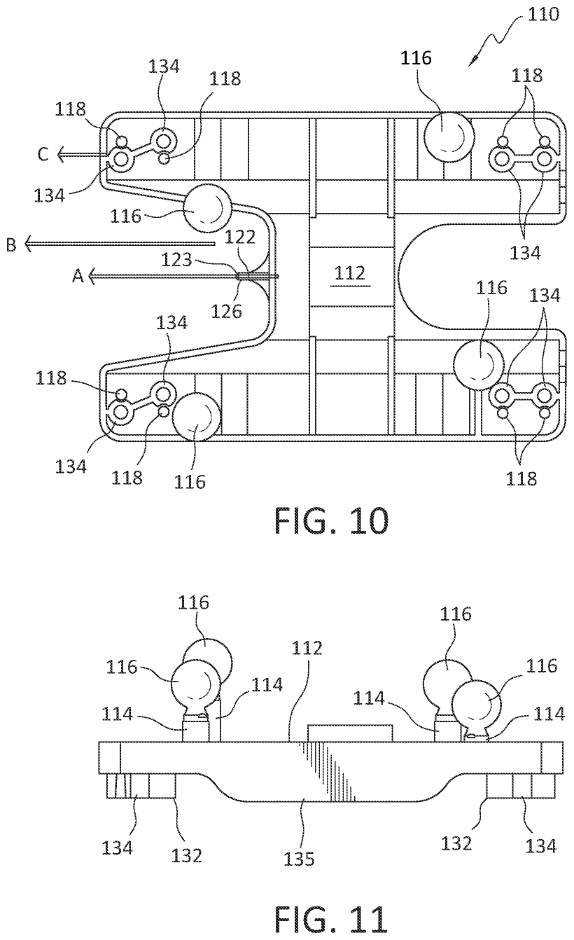

As shown in FIG. 1, pointer extension 26, which terminates in a pointer tip 27, is attachable to the device 10 via a pointer extension post 22. The pointer extension 26 may be any length needed to carry out the needed calibration. The pointer extension post 22 may be integral with the body 12 or be one or more separate components mounted by any method including being attached by snap fit, threaded fit, a separate fastener, or a collet feature. The pointer extension post 22 may be of any length and cross section and may be located at various positions on the body 12, including for example, any of the arrow locations A, B, and C (illustrated in FIG. 10). The pointer extension post 22 terminates in a tip 23. When the device 10 is used without a pointer extension 26, this allows for a relatively short pointer that stays out of the way for imaging. When the device 10 is used with pointer extension 26, this allows for a longer pointer which may be more convenient for the surgeon during registration when the tip 27 is being touched to surgical instruments, for example. Pointer extension 26 may be mounted to pointer extension post 22 by any method including being attached by snap fit, threaded fit, a separate fastener, or a collet feature. Pointer extension 26 may be of any shape or size and any length. Multiple pointer extensions 26 may be provided with different lengths. In such a case, the CAS system would be configured with the dimensions of the extensions and provisions for informing the CAS system which extension is being used would be provided. A variety of pointer tip shapes and types may be be used.

Body 12 forms a conical divot 28 terminating in a point and forms a cylindrical divot 29 terminating in a plane. Conical divot 28 provides a point for a complementary surgical instrument feature to engage during calibration (or calibration verification). Cylindrical divot 29 provides a flat planar surface for a complementary instrument feature to engage during calibration (or calibration verification). For example, an end of a medical instrument with its own navigation array may be brought into contact with the conical divot 28 or flat divot 29. The location and orientation of the conical divot 28 and flat divot 29 relative to the reflective spheres 16, fiducials 18, pointer tip 27 are known and thus the CAS system can use this known relationship in calibration (or calibration verification) of the surgical instrument.

A number of mounting bores 34 can be provided for attachment to an arm or other component of a medical device. The device 10 may be attached to a patient using double sided tape or other fixation methods.

Ears 35 are shown on the lateral sides of the body 12 to assist in handling the device 10. While the ears 35 are shown as flat structures extending away from the lateral sides of the body 12, the ears 35 may take other shapes and sizes.

The reflective spheres 16, the fiducials 18, pointer tip 27 (possibly extended by a pointer extension), and/or divots 28, 29 are held fixedly in relative position to each other by the body 12. The system 10 accordingly provides a multiple featured device 10. Any combination of these features may be used.

The body 12 may be made of injection molded plastic, preferably at least partially made of a radiolucent, such, as for example, injection molded plastic or machined polymer. The posts 14 may be integral with the body 12, or separate from the body 12, and also made of injection molded plastic such as machined polymer. If the posts 14 are made separate from the body 12 then they may be made of metal, for example, stainless steel, titanium, aluminum, etc. The reflective spheres 16 may be molded or machined plastic with reflective coating, paint, or tape. The pointer extension post 22 may be made integral with the body 12, or separate from the body 12, and also made of, for example injection molded plastic, machined plastic, stainless steel, titanium, or the like. The pointer extension 26 may be made of injection molded plastic, machined plastic, stainless steel, titanium, or the like. The fiducials 18 may be made of stainless steel, titanium, ceramic and/or tantalum or any other radiopaque material.

The CAS system may use the manufacturing specifications for the locations of reflective spheres 16, fiducials 18, pointer tip 27, and conical divot 28 and flat divot 29. However, because of manufacturing tolerances, there will be variations in the actual as-manufactured locations of the reflective spheres 16, fiducials 18, pointer tip 27, conical divot 28, and flat divot 29. Therefore, after manufacturing, device 10 may be accurately measured to determine the actual locations of the reflective spheres 16, fiducials 18, pointer extension post 22, tip 27, flat divot 29, and conical divot 28. This as-manufactured measurement data may be used by the CAS system instead of the manufacturing specifications to achieve improve accuracy.

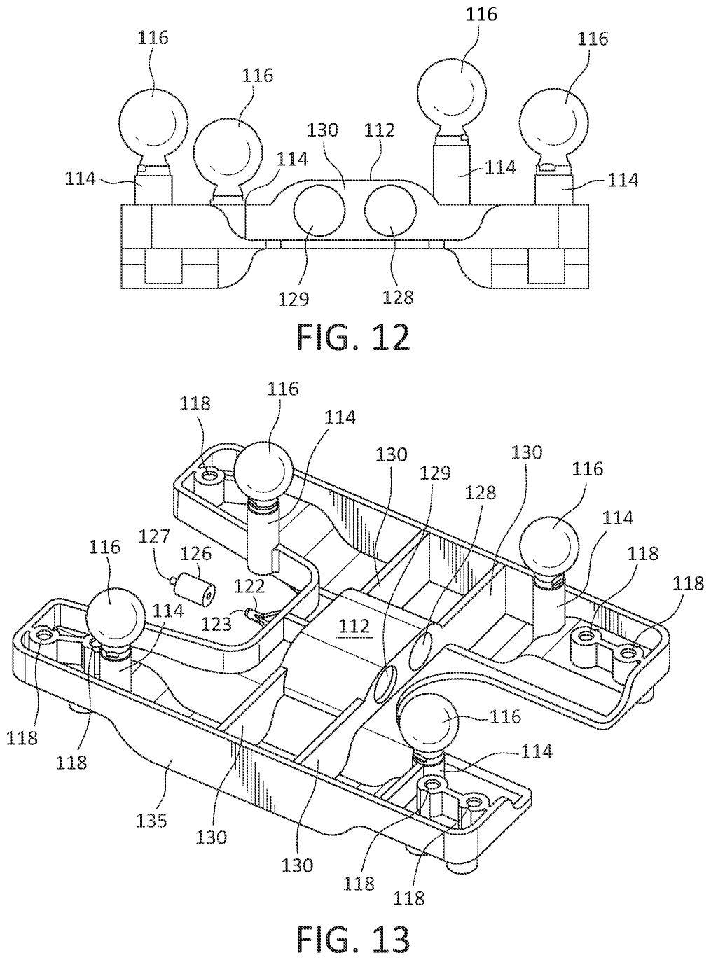

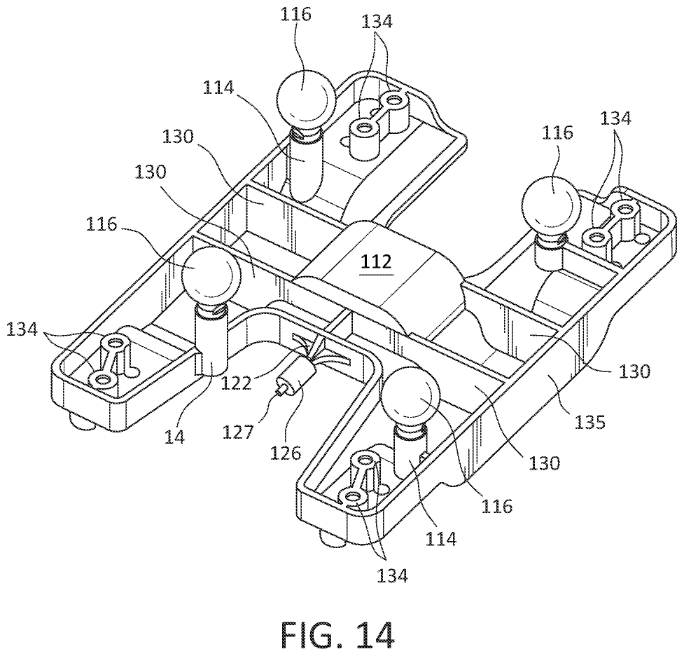

FIGS. 10 through 14 illustrate another embodiment of a device for use in computer-aided surgery.

FIGS. 10 through 14 illustrate a device 110 including a body 112. The body 112 has four posts 114, with each respective post supporting a reflective sphere 116. The reflective spheres 116 are optically visible to a spatial camera. The reflective spheres 116 thus provide an optical element. As described above, the number of optical elements may vary as well as the type of optical element may vary.

Although the posts 114 are shown as elongated conical cylinders, they may be of any shape, and have different lengths from each other. The posts 114 may simply be mounting locations on the body 112.

The posts are injection molded with the body 112. Although illustrated as being injection molded to be integral with the posts 114, the posts 114 may be separate components mounted by any method including being attached by snap fit, threaded fit, a separate fastener, or a collet feature. If the posts 114 are made separate from the body 112 then they may be made of metal, for example, stainless steel, titanium, aluminum, etc. Although the posts 114 are shown as elongated conical cylinders, they may be of any shape, and have different lengths from each other. The posts 114 may simply be mounting locations on the body 112.

The posts 114 each support a respective optically reflective sphere 116. The reflective spheres 116 are optically visible to a spatial camera.

The reflective spheres 116 form an optically visible array. The reflective spheres 116 are shown as being threadably connected to the end of a respective post 114, but other attachment mechanisms and methods may also be used. As described above, the purpose of the respective reflective spheres 116 is to be visible to a camera, using visible or infrared light. As described above, the reflective spheres 116 alternatively may be replaced by LEDs. The purpose of the reflective spheres 116 is to be visible to a location device such as a spatial camera, to determine locations of the optical array, and then the CAS system can in turn determine the locations of various features of device (as an alternative the CAS system and the camera system can be a single computing device or they may be separate devices that communication with each other). Any or all of the different posts 114 may be of different lengths from each other or the same length. The embodiment of FIG. 1 shows the posts 14 being in a common plane, i.e., the posts 14 are of the same length so they are substantially in a plane. The embodiment of FIG. 11 shows posts that have varying heights which places the spheres in positions such that they are not in a common plane.

Also mounted to the body 112 are a plurality of fiducials 118. These are attached to the body using a snap fit, fasteners or may be molded into the body 112. The fiducials 118 are radiopaque and are visible during a C-arm X-ray scan of the body 112 and the patient. The fiducials 118 may be separate components mounted by any method including being attached by snap fit, threaded fit, a separate fastener, or a collet feature. The fiducials 18 may be made of stainless steel, titanium, ceramic and/or tantalum or any other radiopaque material.

The body 112 also supports a pointer extension post 122 with a pointer tip 123. The pointer extension post 122 may be integral with the body 112 or be one or more separate components mounted by any method including being attached by snap fit, threaded fit, a separate fastener, or a collet feature. The pointer extension post 122 may be of any length and cross section and may be located at various positions on the body 112, including, for example, any of the arrow locations A, B, and C. Differing pointer extensions 126 may have different lengths from each other and each terminate in a tip 127. This allows for a relatively short pointer extension post 122 that stays out of the way for imaging while supporting a longer pointer for accuracy checks. The pointer extension 126 can be mounted by any method including being attached by snap fit, threaded fit, a separate fastener, or a collet feature. The pointer extension post 122 can support a measuring extension 126 that is snap fit, threaded fit, attached by a fastener, or the like, to pointer extension post 122. If a pointer extension 126 is used in the procedure, the user may specify to the CAS system that a pointer extension is being used (in such case where only one length extension is provided) or the user may specify to the CAS system which pointer extension is being used (in such case where multiple length extensions are available) is being used (i.e., indicating the length and location of the measuring tip) using various methods. One approach is for the user to input information regarding the measuring tip 126 used via a graphical user interface (GUI). Another approach would be to have the tip 127 of the measuring tip 126 placed in a divot on another instrument or navigation array that has a known location. Further, machine vision recognition or other techniques as noted above may be utilized.

Body 112 forms a conical divot 128 and/or a cylindrical or flat divot 129. The conical divot 128 terminates in a point. The cylindrical divot terminates in a flat plane. The point and/or the flat plane provide a surface for a complementary instrument feature to engage to use during calibration. The divots 128 and 129 have the same functionality as described above with respect to body 112.

The rear or other side of the body 112 may be partially hollow and supported by webs 130.

A number of mounting bores 134 can be provided on the body 112 for attachment to an arm or other component of a medical device. The system 110 may be attached to a patient using double sided tape or other fixation methods.

Ears 135 are shown on the lateral sides of the body 112 to assist in handling the device 110. Bodies 132 are shown for attachment to the patient using double sided tape or other adhesive methods.

The reflective spheres 116, the fiducials 118, and the extension post 122 are held fixedly in relative position to each other by the body 112. The system 110 accordingly provides a multi-feature device. Any of these features may be used in any combination.

While each of the embodiments are described above in terms of their structural arrangements, it should be appreciated that the invention also covers the associated methods of using the embodiments described above.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications and combinations of the various embodiments can be affected while remaining within the spirit and scope of the invention. Accordingly, the foregoing disclosure, description, and figures are for illustrative purposes only and do not in any way limit the invention, which is defined only by the claims.

What is claimed is:

1. A device for computer aided surgery, comprising:
a body;
an optical element coupled to the body;
a fiducial coupled to the body; and
a calibration element.

2. The device of claim 1, wherein the device comprises at least three optical elements.

3. The device of claim 1, wherein the device comprises at least three fiducials.

4. The device of claim 1, wherein the calibration element is a calibration divot forming an indentation on the body configured to receive a surgical instrument.

5. The device of claim 4, wherein the calibration divot further comprises a flat cylindrical positioning divot.

6. The device of claim 4, wherein the calibration divot further comprises a conical positioning divot.

7. The device of claim 1, wherein the calibration element is a pointer tip extending from the body.

8. The device of claim 7, wherein the pointer tip is adapted to receive a first pointer extension.

9. The device of claim 7, wherein the pointer tip is removable from the body.

10. The device of claim 7, wherein the pointer tip is affixed to a pointer extension on the body.

11. A system comprising the device of claim 8 and further comprising a second pointer extension having a different length than the first pointer extension.

12. A system comprising the device of claim 1 and further comprising data representing an as-manufactured dimension of the device.

13. A device for computer aided surgery, comprising:
a body;
an optical element, wherein the optical element is configured to be visible to a location camera; and
a fiducial, wherein the fiducial is radiopaque; and
a calibration element,
wherein the optical element, the fiducial, the body, and the calibration element are held in relation to each other in a fixed position.

14. The device of claim 13, wherein the calibration element is a calibration divot forming an indentation on the body configured to receive a surgical instrument.

15. The device of claim 14, wherein the calibration divot further comprises a flat cylindrical positioning divot.

16. The device of claim 14, wherein the calibration divot further comprises a conical positioning divot.

17. The device of claim 13, wherein the calibration element is a pointer tip mounted to the body.

18. The device of claim 17, wherein the pointer tip is configured to receive a first pointer extension.

19. The device of claim 17, wherein the pointer tip is removable from the body.

20. The device of claim 17, wherein the pointer tip is affixed to a pointer extension on the body.

21. A system comprising the device of claim 17 and further comprising a second pointer extension having a different length than the pointer extension.

22. A system comprising the device of claim 13 and further comprising data representing an as-manufactured dimension of the device.
